(19) United States
(12) Patent Application Publication
(10) Pub. No.: US 2006/0083800 A1
(43) Pub. Date: Apr. 20, 2006

(54) Composition for the treatment of malaria

(76) Inventor: Som C. Pruthi, Monterey, CA (US)

Correspondence Address: Robert M. Downey;ROBERT M. DOWNEY, P.A., Suite 300, 601 South Federal Highway, Boca Raton, FL, 33432, US

(21) Appl. No.: 10/968,373

(22) Filed: Oct. 18, 2004

(51) Int. Cl. A61K 36/185

(52) U.S. Cl. 424/769; 424/776; 424/773

(57) ABSTRACT

A composition for use in treating malaria includes: 4%-38% fever nut by weight; 24%-89% kutaki by weight; 3%-28% five-leaved chaste tree by weight; and 3%-26% karchi by weight.

### BACKGROUND OF THE INVENTION

1. Field of the Invention

The present invention relates to a composition comprising all natural ingredients and, more particularly, to an all natural composition for the treatment of malaria.

2. Discussion of the Related Art

Malaria is one of the world's deadliest infectious diseases and is particularly prevalent in tropical and sub-tropical regions. Caused by a parasite, plasmodium, and transmitted by mosquitos, malaria remains to be a major health concern to the world population. About 170 species of plasmodium exist, but only four cause the disease malaria in humans. The species P. Falciparum produces the most severe symptoms of malaria and is responsible for most malaria related deaths.

Malaria may be transmitted by the bite of an infected mosquito, a blood transfusion using contaminated blood or by injection with a needle that was previously used by an infected person. Typically, mosquitos pick up the malaria parasite from the blood of an infected person when feeding. The parasite is then injected into another person when the mosquito bites.

Inside the person, the parasite multiplies and invades the person's red blood cells. Parasites may remain in the blood stream for years causing repeated attacks of malaria. Symptoms of malaria include fever and flu-like illness, including shaking chills, headache, muscle aches and tiredness. Symtoms usually begin 10-35 days after a mosquito injects the parasite. Malaria is diagnosed by identification of the parasite in a blood sample.

Malaria occurs in over 100 countries, mostly in Africa, Asia, South America and Central America. Over 40% of the world's population is at risk. The World Health Organization estimates that 300 to 500 million malaria related cases occur annually. Malaria causes an estimated 2.7 million deaths per year, mostly children under the age of 5 years. About 10% of hospital admissions and 20-30% of doctor's visits in Africa are attributed to malaria, placing a heavy burden on the health care system. Spread of malaria due to international travel is a serious concern for the developed countries.

Various medications are presently used for the treatment of malaria. However, many of these medications are costly and are not always available in certain parts of the world. Moreover, some medications used to treat malaria have been found to be dangerous and possibly fatal. Other medications are known to have undesirable side effects. The most common drug for treating malaria is chloroquine. Other drugs include melfloquine, doxycycline, hydroxychloroquine, halofantrine and primaquine. Drug choice often depends on one of the four types of malaria parasites. Parasite resistance to these drugs is a growing problem.

During the mid-1900's, malaria appeared to have been brought under control through the use of various medications and insecticides. However, evolving strains of drug resistant parasites and insecticide resistant mosquitos have lead to a resurgence of the disease, particularly in the tropical and sub-tropical regions. Presently, no vaccine is available for preventing infection.

For the reasons advanced above, there remains an urgent need in the world health community for an economical and highly effective treatment for malaria. More particularly, there remains a definite need for an inexpensive and safe product which can be used worldwide for the treatment of persons infected with malaria. The present invention provides an affordable composition comprising all natural ingredients which is effective in treating malaria throughout the world.

### SUMMARY OF THE INVENTION

The present invention is directed to a composition for the treatment of malaria without toxicity. The composition has been shown to be very effective in treating the malaria infection in 21 days of the therapy. Some improvement may be evident in ten days. The compositon is comprised of four natural ingredients including fever nut, kutaki, five-leaved chaste tree and karchi.

In a preferred embodiment, the composition is manufactured in the form of a powder as capsules, tablets, syrup or in a liquefied form for subcutaneous injection.

### DETAILED DESCRIPTION OF THE PREFERRED EMBODIMENTS

The composition includes the combination of fever nut seed kernels, kutaki rhizomes, five-leaved chaste tree roots and karchi seeds. According to the composition of the present invention, Fever Nut in the amount of between 4%-38% by weight of the composition; kutaki in the amount of between 24%-89% by weight of the composition, five-leaved chaste tree in the amount of between 3%-28% by weight of the composition and karchi in the amount of between 3%-26% by weight of the composition.

Fever nut, botanically known as Caesalpinia Bonduc, is a climbing shrub common throughout India near seacoasts. In a preferred embodiment, seed kernels are used in the composition.

Kutaki is botanically known as Picrorrhiza Kurrooa. Kutaki grows on the northwestern Himalayas from Kashmir to Sikkim. Kutaki rhizomes are used in this composition.

Five-leaved chaste tree is known botanically as Vitex Negundo and is found in southern India and Bengal. Roots of the five-leaved chaste tree are used in the present invention.

Karchi is known botanically as Holarrhena Antidysentrica. It is a small tree indigenous to the tropical Himalayas. Seeds from the karchi tree are used in the present invention.

All four ingredients have been in use for thousands of years to fight different diseases.

The composition of the present invention is prepared by grinding fever nut seed kernels into a fine powder in gradual steps to avoid excessive heating. Excessive grinding may result in overheating and destroying the natural properties of the ingredient. Therefore, it is best to grind in series of steps, wherein smaller particles are separated from the remainder of the particles by screening. The smaller particles of a predetermined maximum size are further ground and again separated, repeating the process in a series of steps to produce a fine powder. In a preferred embodiment, the process of grinding is accomplished with the use of a mortar and pestle. A series of sieves, each having predetermined sized openings in it screen, are used to separate the ground material throughout the process.

Kutaki rhizomes are broken in small pieces and ground in three steps to a fine powder similar to the various steps outlined above to avoid excessive heating at any time in the process of grinding. Commercially available grinding techniques may also be used for mass production but care should be taken to avoid excessive heating.

Five-leaved chaste tree roots are also ground gradually similar to the process outlined above.

Karchi seeds are used in this invention. Gradual grinding is done in steps as mentioned above to keep the natural healing power of the herbs.

After all four powders are ground and sieved in steps to a desired mesh size, the powders are placed in a mixer chamber (e.g. bowl or container) in accordance with their predetermined percentages by weight. The four powders are then mixed at slow speed, by stirring, until a homogeneous blend is achieved. The homogeneous blend is kept in a cool dry place until manufactured in the form of capsules, tablets, syrup or other forms, in accordance with manufacturing techniques well known in the field.

The capsules, incorporating the composition of the present invention, are preferably of a concentration of between 450-900 mg. In capsule form, this may require taking one to two capsules per day, depending upon the concentration of each capsule and the prescribed dosage. The optimum dosage for most patients seems to be 450 mg per day. One capsule should be taken orally in the morning. In chronic cases, one capsule in the morning and one capsule in the evening will be required. Improvement will ordinarily be evident in 10 to 15 days from the beginning of the treatment. Treatment should be continued for at least three weeks. During the treatment period, alcohol, meat, drugs (other than prescribed by a physician) spices, sour juices and fried foods should be avoided.

In order to verify the efficacy of the composition of the present invention in treating malaria, several test studies were performed. The typical results of the three patients are set forth below:

Dosage: 1 capsule a day of 450 mg total weight daily for 10-21 days.

Clinical Study Examples

| | Hemoglobin | Malaria |
|---|---|---|
| Patients | (H.b: gm/dl) | (type) |
| Male, Age 32 | 7.5 | P. Vivax |
| Female, Age 19 | 9.5 | P. Vivax |
| Male, Age 56 | 8.5 | P. Falciparum |

Laboratory test results were in the normal range of 11 to 18 gm/dl in all three cases. None of the patients had chills or fever after the treatment. There were no ill effects.

While the instant invention has been shown and described in accordance with a preferred and practical embodiment thereof, it is recognized that departures from the instant disclosure are contemplated within the spirit and scope of the present invention which, therefore, should not be limited except as set forth in the following claims as interpreted under the doctrine of equivalents.

What is claimed is:

1. A composition for the treatment of Malaria comprising the following ingredients:
seed kernels of fever nut in an amount of between 4% and 38% by weight of the composition;
kutaki in an amount of between 24% and 89% by weight of the composition;
roots of five-leaved chaste tree in an amount of between 3% and 28% by weight of the compositon;
seeds of karchi in an amount of between 3% and 26% by weight of the composition.

2. A composition for the treatment of Malaria comprising the following ingredients:
powder of fever nut derived from the seed kernels in an amount of between 4% and 38% by weight of the composition;
powder of Kutaki derived from rhizomes in an amount between 24% and 89% of the weight of the compositin;
powder of five-leaved Chaste Tree derived from roots of the tree in an amount between 3% and 28% of the weight of the composition;
powder of Karchi derived from seed in an amount between 3% and 26% of the weight of the composition.

3. The composition as recited in claim 2 wherein said composition is contained in a capsule form comprising between 450 mg. and 900 mg. of said composition.

4. The composition as recited in claim 2 wherein said composition is contained in a tablet form comprising between 450 mg. and 900 mg. of said composition.

5. The composition as recited in claim 2 wherein said composition is contained in a syrup form for oral consumption in an amount of between 450 mg and 900 mg of said composition.

6. The composition as recited in claim 2 wherein said composition is contained in a liquid form for subcutaneous injection, wherein the composition is administered in dosage of between 450 mg and 900 mg.
